(19) United States
(12) Patent Application Publication
(10) Pub. No.: US 2010/0130599 A1
(43) Pub. Date: May 27, 2010

(54) CYP2C9*8 Alleles Correlate With Decreased Warfarin Metabolism And Increased Warfarin Sensitivity

(76) Inventors: William COTY, Alhambra, CA (US); Larisa Cavallari, Northfield, IL (US)

Correspondence Address: MORGAN, LEWIS & BOCKIUS, LLP (SF), ONE MARKET SPEAR STREET TOWER, SAN FRANCISCO, CA, 94105, US

(21) Appl. No.: 12/572,908

(22) Filed: Oct. 2, 2009

Related U.S. Application Data
(60) Provisional application No. 61/102,469, filed on Oct. 3, 2008.

(51) Int. Cl. A61K 31/352; C12Q 1/68

(52) U.S. Cl. 514/457; 435/6

(57) ABSTRACT

The present disclosure is related to a method of identifying a subject with increased sensitivity to warfarin. The method includes identifying a CYP2C9*8 polymorphism in the subject, wherein the presence of said polymorphism is indicative of a patient with increased sensitivity to warfarin relative to a subject having the corresponding wild-type allele.

### RELATED APPLICATIONS

This application claims priority to U.S. provisional patent application Ser. No. 61/102,469, entitled the same, filed Oct. 3, 2008, and herein incorporated by reference in its entirety.

### GOVERNMENT RIGHTS

Not applicable.

### FIELD OF THE INVENTION

The field relates to pharmacogenomics and genotyping as affects warfarin and other drug metabolism and dosing.

### BACKGROUND OF THE INVENTION

The polymorphic enzyme CYP2C9 metabolizes numerous clinically important drugs, including Warfarin. Different CYP2C9 polymorphisms are responsible for different alleles, of which today there are more than 30 known (see www.cypalleles.ki.se). The alleles include, e.g., *1 (wild-type), *2, *3, *4, *5, *6, *7, *8, *9, *10, *11, *12 and *14. Various of the foregoing alleles result in enzymes that exhibit more or less in vivo activity and clearance of drug substrates relative to the wild-type enzyme. The differences can have dire effects on patients. For example, the anticoagulant, Warfarin, is the 11^{th} most prescribed drug in the United States and estimated to be responsible for 15% of all severe adverse events (second only to Digoxin). Lazarou et al. (1998) JAMA 279: 1200-1205. Too little Warfarin can lead to clotting and too much can lead to bleeding.

The most prevalent CYP2C9 polymorphisms in the Caucasian population are CYP2C9*2(430 C>T) and *3 (1075 A>C) and carry allelic frequencies of approximately 12% and 7%, respectively. Those alleles are much less common among African-Americans, where other alleles, including *8, are more prevalent.

The *8 allele is characterized by a 449G>A polymorphism in the cDNA sequence, which gives rise to an R150H change in the amino acid sequence of the enzyme that metabolizes warfarin. Despite the molecular characterization of the *8 polymorphism, its downstream effect on Warfarin metabolism has heretofor remained unknown.

Known is that *8 has little effect on in vivo metabolism of Losartran, an angiotensin II receptor antagonist drug used mainly to treat high blood pressure (Allabi et al, Clinical Pharmacology & Therapeutics, vol. 76, no. 2, pp. 113-118 (2004)), increases metabolism in vitro of Tolbutamide, an oral hypoglycemic drug used to treat type II diabetes (Blaisdell et al., Pharmacogenetics, vol. 14, no. 8, pp. 527-537 (2004)), and decreases in vivo metabolism of phenytoin, an anti-epileptic drug (Allabi et al., Pharmacogenetics and Genomics, vol. 15, no. 11, pp. 779-786 (2005)). The *8 polymorphism therefore appears to exert its effect in a substrate-specific and dependent manner, and the results heretofor have been mixed and unpredictable. Accordingly there is a need for identification of the functional consequences of the *8 polymorphism in warfarin treatment.

### SUMMARY OF THE INVENTION

It is an object of the invention to improve dosing and safety for warfarin recipients and prospective recipients based on the finding that those who possess a CYP2C9*8 allele, either in homozygous or heterozygous form, or in a compound heterozygous form with another reduced-activity allele, require less warfarin relative to those who are homozygous for the wild-type allele (*1/*1).

Accordingly, in a first aspect the invention features a method of administering warfarin to a subject by determining whether the subject possesses a CYP2C9*8 allele, and if so, administering a lower amount of warfarin to the subject than were the subject homozygous wild type (*1/*1).

In some embodiments the subject is homozygous for the *8 allele. In other embodiments, the subject is heterozygous, e.g., *1/*8, and in still another embodiment, the subject is compound heterozygous for *8 and another CYP2C9 allele which potentially confers lower-enzyme activity; e.g., *5, *6 and/or *11.

In some embodiments the method features administering 55% to 85% of the warfarin dose over time that would be administered to a homozygous wild type patient based on observed reduced need.

In some embodiments the method features administering 25 mg/week to 40 mg/week to a subject upon determination that the subject possesses the reduced requirement signified by the presence of the *8 allele.

The patent/recipient is a human, most preferably an African-American or black African.

In another aspect, the invention features a warfarin dosing algorithm, said warfarin dosing algorithm comprising one more mathematical operations that consider CYP2C9*8 genotype in calculating, predicting, and/or prescribing warfarin dosage to a patient, and wherein said calculating, predicting, and/or prescribing references a lower amount of warfarin use relative to a homozygous wild type CYP2C9 genotype.

The present invention is directed to molecules and methods useful for determining the identity of the *8 polymorphic site in the CYP2C9 gene and correlating the identity of such site with a decreased warfarin metabolism and increased warfarin sensitivity. The invention is particularly concerned with a genetic predisposition for decreased warfarin metabolism and therefore, increased sensitivity.

The invention also provides a kit, suitable for genetic testing. Such a kit may contain primers for amplifying regions of CYP2C9 gene encompassing regions where at least the *8 polymorphism is found. The primers may but need not be allele-specific. The kit may also contain complementary capture probes and/or signal probes for use in sandwich assays, and sources of control target polynucleotides as positive and negative controls. Such sources may be in the form of patient nucleic acid samples, cloned target polynucleotides, plasmids or bacterial strains carrying positive and negative control DNA.

In one aspect, the invention provides an oligonucleotide for determining the identity of a polymorphic site of a CYP2C9 molecule of a target polynucleotide, wherein: a) said target polynucleotide comprises a segment of CYP2C9; b) said segment comprises said polymorphic site; and c) said oligonucleotide is complementary to said segment.

The invention particularly concerns the embodiments wherein said oligonucleotide comprises said polymorphic site, and said oligonucleotide is an allele-specific oligonucleotide or wherein said oligonucleotide does not comprise said polymorphic site, and said oligonucleotide is a primer oligonucleotide.

The invention further concerns the embodiment in which such oligonucleotide is labeled with a label selected from the group: radiolabel, fluorescent label, bioluminescent label, chemiluminescent label, nucleic acid, hapten, or enzyme label.

The invention further provides a primer oligonucleotide for amplifying a region of a target polynucleotide, said region comprising a polymorphic site of CYP2C9 wherein said primer oligonucleotide is substantially complementary to said target polynucleotide, thereby permitting the amplification of said region of said target polynucleotide.

In another aspect, the invention provides methods of predicting relative sensitivity to warfarin of a patient, where a sample comprising a polynucleotide encoding CYP2C9 molecule or fragment of the polynucleotide from the subject is obtained and the sample is analyzed for a polymorphic site at nucleotide position 449 of the polynucleotide or fragment of the polynucleotide, wherein a polypeptide with a histidine at position 150 is produced and indicates a decreased metabolism of warfarin, thereby providing an indication of the therapeutically effective dose of warfarin for the patient.

As such, the invention encompasses methods in which proteins or nucleic acids are analyzed to identify the polymorphism. That is, when nucleic acids are analyzed, a G to A mutation is identified at nucleotide position 449 of the CYP2C9 gene (G449A) while an R to H amino acid mutation is identified at amino acid 150 (R150H). Identification of the polymorphism at either the nucleic acid or protein level is predictive of decreased warfarin metabolism or increased warfarin sensitivity as compared to the wild type nucleic acid or protein.

Other aspects and embodiments of the invention will be obvious to the person of ordinary skill in the art, who will appreciate that various embodiments can be modified or combined as appropriate to achieve results consistent with the spirit of the invention. For example, it is anticipated that the *8 allele may affect the metabolism of certain other drugs, including but not limited to drugs that are structurally and/or functionally similar to warfarin, e.g., coumadin derivatives and analogs as known in the literature. See, e.g., Kater et. al., Clinical and Diagnostic Laboratory Immunology (2002) p. 1396-1397; Tummino et al., Biochem Biophys Res Commun. 1994 May 30; 201(1):290-4. U.S. Pat. Nos. 7,285,671, 7,253,208, 7,145,020, and 6,512,005; and published US Patent Applications 20090216561, 20090214496, 20090087856, 20090082430, 20080221204, 20080045686, 20080027132, 20070129429, 20070093744, 20060287388, 20050245603, 20040220258, 20040091937, and 20020120155. As referenced herein, derivatives and analogs shall be defined synonymously with one another.

### BRIEF DESCRIPTION OF THE DRAWINGS

Not applicable.

### DETAILED DESCRIPTION OF THE PREFERRED EMBODIMENTS

Applicants herein report experimentation and results demonstrating, for the first time, that the enzyme coded by the *8 allele exhibits decreased activity toward warfarin as its substrate. This bodes more accurate and safe dosing of warfarin patients with the *8 allele, along with attendant cost savings insofar as emergency room visits and hospitalizations can be reduced by avoiding over-dosing of patients with warfarin. Significantly, subsequent to the priority date accorded this application, work by an independent group seems to corroborate Applicants' surprising findings. See Scott et al., CYP2C9*8 is prevalent among African-Americans: implications for pharmacogenetic dosing, Pharmacogenomics, (2009) 10(8), 1243-1255.

### I. DEFINITIONS

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3rd Ed.” Vols. A and B, Plenum Press, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology, within the skill of the art. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity.

As used herein, the terms “label”, “detectable label”, and “reporter molecule” refer to a molecule capable of being detected, including, but not limited to, radioactive isotopes, fluorescers, chemiluminescers, chromophores, magnetic resonance agents, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising warfarin disclosed herein required to provide a clinically significant decrease in clotting, for example.

As used herein, the terms “treat” or “treatment” are used interchangeably and are meant to indicate a postponement of development of disease and/or a reduction in the severity of such symptoms that will or are expected to develop. The terms further include ameliorating existing symptoms, preventing additional symptoms, and ameliorating or preventing the underlying metabolic causes of symptoms.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

In the present invention the phrase “stringent hybridization conditions” or “stringent conditions” refers to conditions under which a nucleic acid will hybridize to its target sequence, but to a minimal number of other sequences. Stringent conditions are sequence-dependent and will be different in different circumstances and in the context of this invention. Low stringency hybridization and annealing conditions permit the annealing of complementary nucleic acids that contain mismatched nucleic acids. As the stringency is raised, annealing of sequences containing mismatched nucleic acids is disfavored. Conditions which result in low or high stringency levels are known in the art (e.g., increasing the annealing temperature raises the stringency). Hybridizations are usually performed under stringent conditions, for example, at a salt concentration of no more than 1M and a temperature of at least 25° C. For example, conditions of 5×SSPE (750 mm NaCl, 50 mM NaPhosphate, 5 mM EDTA, pH 7.4) and a temperature of about 25° C. to 30° C. are suitable for allele-specific probe hybridizations.

“Homology” refers to the percent similarity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence similarity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Readily available computer programs can be used to aid in the analysis of homology and identity. Such methods for determining homology also may be used to align similar sequences and so identify corresponding positions in two or more sequences (nucleic acid or polypeptide sequences). The two or more sequences may represent splice variants or homologous sequences from different species. While the polymorphisms of the present invention have been described by reference to the coding sequence of particular molecules such as, e.g., the human CYP2C9 gene as described in www.cypalleles.ki.se (containing links to GenBank Accession Nos and citations affiliated with the wildtype and mutant gene, mRNA and peptide sequences for CYP2C9), one of ordinary skill will readily recognize that the invention is intended to encompass polymorphisms occurring in linked or corresponding positions in different sequences.

The term “wild type” as used herein in reference to a gene, nucleic acid or gene product, especially a protein and/or biological property, denotes a gene, gene product, protein, or biological property predominantly found in nature.

The term “polymorphism” as used herein refers to the occurrence of two or more genetically determined alternative sequences or alleles in a population. A single nucleotide polymorphism occurs at a polymorphic site occupied by a single nucleotide, which is the site of variation between allelic sequences. A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

The term “allele-specific oligonucleotide” refers to an oligonucleotide that is able to hybridize to a region of a target polynucleotide spanning the sequence, mutation, or polymorphism being detected and is substantially unable to hybridize to a corresponding region of a target polynucleotide that either does not contain the sequence, mutation, or polymorphism being detected or contains an altered sequence, mutation, or polymorphism.

### II. OVERVIEW

The present invention discloses methods, compositions, and kits for determining sensitivity to warfarin.

In one aspect, the invention relates to methods and compositions for the treatment and diagnosis of clotting and bleeding disorders. In particular, the present invention identifies and describes polymorphic variations in the human CYP2C9 gene at nucleotide 449 of the coding region. In particular, the variation is a G to A substitution at nucleotide 449. The resulting polypeptides have either an Arg (R) in the wild-type or His (H) in the variant at amino acid at position 150. The polymorphic variation can be used to assess the risk of clotting disorders and importantly, the level of warfarin treatment to be administered. In addition, the variation can be used for the identification of subjects having increased warfarin sensitivity or decreased warfarin metabolism, which informs how much warfarin should be administered to the subject.

### III. POLYMORPHISMS OF THE PRESENT INVENTION

The particular gene sequences of interest to the present invention comprise “mutations” or “polymorphisms” in the genes for the CYP2C9.

CYP2C9 is cytochrome P450 2C9. CYP2C9*8 refers to polymorphisms in the nucleic acid or amino acid sequence of a CYP2C9 gene or gene product. For the purposes of identifying the location of a polymorphism, the first nucleotide of the start codon of the coding region; (the adenine of the ATG in a DNA molecule and the adenine of the AUG in an RNA molecule) of the CYP2C9 gene is considered nucleotide “1.” Similarly, the first amino acid of the translated protein product (the methionine) is considered amino acid “1.”

As appreciated by one of skill in the art, nucleic acid assays for the detection of polymorphisms are well known. For example, assays are described in US PGPuB 20030207295, incorporated herein by reference. Additional assays for detection of polymorphisms as disclosed herein are described and referenced in the examples below.

In addition to traditional nucleic acid or polypeptide sequencing and nucleic acid hybridization-based techniques, including SNP assays, mass spectroscopy may be used to determine the presence or absence of polymorphisms. This is because the structure of molecules, such as peptides, proteins, receptors, antibodies, oligonucleotides, RNA, DNA, and other nucleic acids such as RNA/DNA hybrids, oligosaccharides, organic molecules and inorganic molecules, can be obtained using mass spectrometry. The mass spectrometry method can provide not only the primary, sequence structure of nucleic acids, but also information about the secondary and tertiary structure of nucleic acids, RNA and DNA, including mismatched base pairs, loops, bulges, kinks, and the like. The mass spectrometric techniques that can be used in the practice of the present invention include MSn (collisionally activated dissociation (CAD) and collisionally induced dissociation (CID)) and infrared multiphoton dissociation (IRMPD). A variety of ionization techniques may be used including electrospray, MALDI and FAB. The mass detectors used in the methods of this invention include FTICR, ion trap, quadrupole, magnetic sector, time of flight (TOF), Q-TOF, and triple quadrupole.

Electrospray ionization mass spectrometry (ESI-MS) is broadly applicable for analysis of macromolecules, including proteins, nucleic acids, and carbohydrates (Crain et al., Curr. Opin. Biotechnol. 9:25-34 (1998)). Fourier transform ion cyclotron resonance mass spectrometry (FT-ICR MS) can be used to resolve very small mass differences providing determination of molecular mass (Marshall, et al., Mass Spectrom. Rev. 17:1-35 (1998)). In addition, Matrix-Assisted Laser Desorption/Ionization Mass Spectrometry (MALDI-MS) is another method that can be used for studying biomolecules (Hillenkamp et al., Anal. Chem. 63:1193 A-1203A (1991)). In MALDI-MS high molecular weight biomolecules are ionized with minimal concomitant fragmentation of the sample material via the incorporation of the sample to be analyzed into a matrix that absorbs radiation from an incident UV or IR laser. This energy is then transferred from the matrix to the sample resulting in desorption of the sample into the gas phase with subsequent ionization and minimal fragmentation. MALDI spectra are generally dominated by singly charged species. Typically, the detection of the gaseous ions generated by MALDI techniques, are detected and analyzed by determining the time-of-flight (TOF) of these ions. While MALDI-TOF MS is not a high resolution technique, resolution can be improved by making modifications to such systems, by the use of tandem MS techniques, or by the use of other types of analyzers, such as Fourier transform (FT) and quadrupole ion traps. Fourier transform mass spectrometry (FTMS, Amster, J. Mass Spectrom. 31:1325-1337 (1996)) can be used to obtain high resolution mass spectra of ions generated by any of the other ionization techniques.

Accordingly, once detected or identified, the polymorphisms of the present invention are preferably used in the diagnosis and/or prognosis of the effectiveness of warfarin treatment. That is, the CYP2C9*8 polymorphism is used in predicting the level of warfarin to be administered to a subject as outlined herein. In a preferred embodiment, the amount of warfarin to be administered to a subject having the CYP2C9*8 allele is from 1-100 mg/week, more preferably 15-60 mg/week, more preferably still 20-50 mg/week, and most preferably 25-40 mg/week. As the person of skill will appreciate, precise amounts may vary as between individuals based on body weight and other factors.

### EXAMPLES

### Summary

A total of 226 African American patients having demographic and clinical parameters as depicted in Table 1 were studied.

TABLE 1
Demographic and clinical characteristics of the African American study

| | All patients |
|---|---|
| Characteristics | (n = 226) |
| Age (years) | 57 ± 15 |
| Female sex | 160 (71) |
| Body surface area (m²) | 2.1 ± 0.3 |
| Past medical history | |
| Venous thromboembolism | 113 (50) |
| Atrial fibrillation or flutter | 42 (19) |
| Stroke or TIA | 73 (32) |
| Heart valve replacement | 15 (7) |
| Hypertension | 162 (72) |
| Diabetes mellitus | 60 (27) |
| Heart failure | 37 (16) |
| Coronary artery disease | 42 (19) |
| Active cancer on chemotherapy | 3 (1) |
| Therapeutic warfarin dose (mg/week) | 40.0 (32.5-55.0) |
| Average INR | 2.5 ± 0.3 |
| Concomitant medications | |
| Aspirin | 64 (28) |
| Simvastatin | 70 (31) |
| Amiodarone | 4 (2) |
| Phenytoin or carbamazepine | 6 (3) |
| Current smoker | 39 (17) |

No. (%), mean ± SD, or median (IQR);
TIA = transient ischemic attack

Height or weight was missing for 2 patients, and concomitant drug therapy was missing for one patient. All other demographic and clinical data were complete. The majority of patients were taking warfarin for secondary prevention of deep vein thrombosis or pulmonary embolism (46%), primary stroke prevention in atrial fibrillation (12%), or secondary stroke prevention (25%) and had a goal INR range of 2 to 3 (90%). The median daily warfarin dose in the study population was 5.7 (range 2.0 to 13.6) mg.

Genotype data were missing for one patient for CYP2C9*3, VKORC1 −4451C>A, and VKORC1497T>G genotypes; two patients for the CYP2C9*6, *8, and *11 genotypes; three patients for CYP4F2 genotype, and nine patients for APOE. All other genotype data were complete. With the exception of the VKORC1 −4451C>A genotype (p=0.01), all genotypes were in Hardy-Weinberg equilibrium. Allele frequencies (Table 2) were similar to those previously reported in African American and black African populations. See Kimmel, S. E. et al. Apolipoprotein E genotype and warfarin dosing among Caucasians and African Americans. Pharmacogenomics J (2007); Scordo, M. G. et al. Genetic polymorphism of cytochrome P450 2C9 in a Caucasian and a black African population. Br J Clin Pharmacol 52, 447-50 (2001); Limdi, N. A. et al. VKORC1 polymorphisms, haplotypes and haplotype groups on warfarin dose among African-Americans and European-Americans. Pharmacogenomics 9, 1445-58 (2008); Allabi, A. C., Gala, J. L. & Horsmans, Y. CYP2C9, CYP2C19, ABCB1 (MDR1) genetic polymorphisms and phenytoin metabolism in a Black Beninese population. Pharmacogenet Genomics 15, 779-86 (2005); Blaisdell, J. et al. Discovery of new potentially defective alleles of human CYP2C9. Pharmacogenetics 14, 527-37 (2004); Marsh, S., King, C. R., Porche-Sorbet, R. M., Scott-Horton, T. J. & Eby, C. S. Population variation in VKORC1 haplotype structure. J Thromb Haemost 4, 473-4 (2006).

TABLE 2
Minor allele frequencies

| | No. variant alleles/ | |
|---|---|---|
| Allele | No. total alleles | Frequency |
| CYP2C9*2 | 10/452 | 0.022 |
| CYP2C9*3 | 3/450 | 0.007 |
| CYP2C9*5 | 3/452 | 0.007 |
| CYP2C9*6 | 6/448 | 0.013 |
| CYP2C9*8 | 29/448 | 0.065 |
| CYP2C9*11 | 8/448 | 0.018 |
| VKORC1 −4451 C > A | 28/450 | 0.062 |
| VKORC1 −1639 G > A | 41/452 | 0.091 |
| VKORC1 497 T > G | 18/450 | 0.040 |
| VKORC1 3730 G > A | 211/452 | 0.467 |
| CYP4F2 V433M | 32/446 | 0.072 |
| APOE ε2 | 50/434 | 0.115 |
| APOE ε3 | 300/434 | 0.691 |
| APOE ε4 | 84/434 | 0.194 |

CYP, cytochrome P450;
VKORC1, vitamin K oxidoreductase complex subunit1;
APOE, apolipoprotein E

Genetic Associations with Warfarin Dose

Compared to those with the CYP2C9*1/*1 genotype, warfarin dose requirements were significantly lower in carriers of a CYP2C9*2, *3, *5, *6, or *11 allele and in those with the CYP2C9*1/*8 or *8/*8 genotype (Table 3).

TABLE 3
Therapeutic warfarin dose by genotype

| | | Median (IQR) | |
|---|---|---|---|
| Genotype | n | dose (mg/week) | p value |
| CYP2C9 | | | |
| *1/*1 | 171 | 42.5 (35.0-56.3) | — |
| *2, *3, *5, *6, or | 28 | 33.1 (28.0-40.0) | <0.001 (versus *1/*1) |
| *11 allele | | | |
| *1/*8 or *8/*8 | 24 | 34.4 (29.5-47.3) | 0.023 (versus *1/*1) |
| Any variant | 52 | 33.8 (28.0-40.6) | <0.001 (versus *1/*1) |
| VKORC1 −4451C > A | | | |
| CC | 200 | 40.0 (32.5-55.0) | 0.119 |
| CA | 22 | 42.5 (31.0-55.6) | |
| AA^{†} | 3 | 28.0 (26.5-30.3) | |
| VKORC1 | | | |
| −1639G > A | | | |
| GG | 189 | 42.5 (33.8-56.3) | 0.002 |
| GA | 33 | 35.0 (27.5-42.5) | |
| AA | 4 | 33.8 (27.8-35.3) | |
| VKORC1 497T > G | | | |
| TT | 208 | 42.0 (32.5-56.1) | 0.027 |
| TG | 16 | 35.0 (27.5-36.3) | |
| GG | 1 | 36.0 — | |
| VKORC1.3730A > G | | | |
| GG* | 60 | 37.5 (30.0-47.5) | 0.171 |
| GA | 121 | 42.0 (34.0-56.3) | |
| AA | 45 | 42.0 (32.5-55.0) | |
| CYP4F2 V433M | | | |
| V/V | 194 | 41.6 (32.5-55.0) | NS |
| V/M | 26 | 40.0 (35.3-51.9) | |
| M/M | 3 | 37.5 (28.8-56.3) | |
| APOE | | | |
| ε2/ε2 | 4 | 35.5 (34.3-38.9) | NS |
| ε2/ε3 | 36 | 40.0 (30.0-49.7) | |
| ε2/ε4 | 6 | 61.3 (38.4-70.0) | |
| ε3/ε3 | 102 | 40.0 (32.1-52.5) | |
| ε3/ε4 | 60 | 43.8 (32.4-56.3) | |
| ε4/ε4 | 9 | 45.0 (40.0-52.5) | |

*VKORC1 3730 GG genotype versus A allele carriers, p = 0.060; CYP, cytochrome P450; VKORC1, vitamin K oxidoreductase complex 1; APOE, apolipoprotein E.

Similarly, doses were lower in individuals with a CYP2C9*5, *6, *8, or *11 allele [n=40, 35.0 (30.0-46.0 mg/wk)] compared to CYP2C9*1 homozygotes (p=0.004). We also observed significant associations between warfarin dose requirements and both the VKORC1 −1639G>A and 497G>T genotypes and a trend toward lower doses with the 373000 genotype versus the non-GG genotype (p=0.06). There was no association between the VKORC1 −4451C>A, CYP4F2 V433M, or APOE genotype and therapeutic warfarin dose. In addition, there was no significant difference in dose between APOE ε4 carriers [45.0 (33.1-56.3) mg/week] versus non-ε4 carriers [40.0 (32.0-52.5) mg/week; p=0.292], between APOE ε4 homozygotes [45.0 (40.0-52.5) mg/week] versus non-c4 homozygotes [40.0 (32.0-55.0); p=0.323], or between those with the APOE ε2/ε4 genotype versus other genotypes (p=0.153).

Regression Analysis of Factors Associated with Warfarin Dose

Clinical variables potentially associated with log-transformed warfarin dose requirements on univariate analysis (as indicated by a p value <0.10) were age, body surface area (BSA), venous thromboembolism, cerebrovascular disease, and use of aspirin or simvastatin (Table 4), but not gender (p=0.99).

TABLE 4
Univariate associations between clinical variables and log warfarin dose

| | Correlation | |
|---|---|---|
| Variable | coefficient | p value |
| Age | −0.341 | <0.001 |
| Body surface area | 0.299 | <0.001 |
| History of DVT or PE | — | 0.024 |
| History of stroke or TIA | — | 0.009 |
| Aspirin use | — | 0.032 |
| Simvastatin use | — | 0.079 |

DVT, deep For the regression model of factors associated with warfarin dose, VKORC1 −1639G > A was the first variable entered (Table 5).

TABLE 5
Factors jointly associated with log warfarin dose requirements

| | | Adjusted | | |
|---|---|---|---|---|
| Entry into | | R² | Standardized | |
| Model | Variable | after entry | Coefficient | p value |
| 1 | VKORC1 −1639G > A | 0.073 | −0.292 | <0.001 |
| 2 | CYP2C9 *2, *3, *5, | 0.150 | −0.309 | <0.001 |
| | *6, *8, or *11 allele | | | |
| 3 | Age | 0.244 | −0.270 | <0.001 |
| 4 | BSA | 0.340 | 0.304 | <0.001 |
| 5 | Stroke or TIA | 0.364 | −0.165 | 0.003 |

VKORC1, vitamin K oxidoreductase complex 1;
CYP2C9, cytochrome P450 2C9;
BSA, body surface area (m²);
TIA, transient ischemic attack

The VKORC1 −1639GA polymorphism alone explained 7.3% of the variability in warfarin dose (adjusted R²). With the addition of the CYP2C9*2, *3, *5, *6, *8 and *11 alleles, the model explained 15% of the overall variance. Once the CYP2C9 and VKORC1 −1639G>A genotypes were entered into the model, none of the other genotypes were significantly associated with warfarin dose requirements. The clinical factors associated with warfarin dose on regression analysis were age, BSA, and cerebrovascular disease. Together, genetic and clinical variables explained 36% of the inter-patient variability in warfarin dose. The addition of gender provided no further contribution to the model (p=0.24). When the CYP2C9*5, *6, *8, and *11 alleles were removed from the model (i.e. treated as *1 alleles), the remaining variables jointly explained 30% of the variability in warfarin dose requirements.

### Discussion

African Americans are underrepresented in pharmacogenomic studies with warfarin. The VKORC1 −1639G>A and CYP2C9*1, *2, and *3 alleles are the only variants included in most published warfarin dosing algorithms and on some commercial genotyping platforms. Anderson, J. L. et al. Randomized trial of genotype-guided versus standard warfarin dosing in patients initiating oral anticoagulation. Circulation 116, 2563-70 (2007); Wen, M. S. et al. Prospective study of warfarin dosage requirements based on CYP2C9 and VKORC1 genotypes. Clin Pharmacol Ther 84, 83-9 (2008); Klein, T. E. et al. Estimation of the warfarin dose with clinical and pharmacogenetic data. N Engl J Med 360, 753-64 (2009); Gage, B. F. et al. Use of pharmacogenetic and clinical factors to predict the therapeutic dose of warfarin. Clin Pharmacol Ther 84, 326-31 (2008); Caldwell, M. D. et al. Evaluation of genetic factors for warfarin dose prediction. Clin Med Res 5, 8-16 (2007); Sconce, E. A. et al. The impact of CYP2C9 and VKORC1 genetic polymorphism and patient characteristics upon warfarin dose requirements: proposal for a new dosing regimen. Blood 106, 2329-33 (2005); Takahashi, H. et al. Different contributions of polymorphisms in VKORC1 and CYP2C9 to intra-and inter-population differences in maintenance dose of warfarin in Japanese, Caucasians and African-Americans. Pharmacogenet Genomics 16, 101-10 (2006). It is clear from the available data that these alleles provide lesser contributions to warfarin dose response in African Americans compared to Caucasians. Limdi, N. A. et al. Influence of CYP2C9 and VKORC1 on warfarin dose, anticoagulation attainment and maintenance among European-Americans and African-Americans. Pharmacogenomics 9, 511-26 (2008); Wang, D. et al. Regulatory polymorphism in vitamin K epoxide reductase complex subunit 1 (VKORC1) affects gene expression and warfarin dose requirement. Blood 112, 1013-21 (2008); Schelleman, H. et al. Warfarin response and vitamin K epoxide reductase complex 1 in African Americans and Caucasians. Clin Pharmacol Ther 81, 742-7 (2007); Gage, B. F. et al. Use of pharmacogenetic and clinical factors to predict the therapeutic dose of warfarin. Clin Pharmacol Ther 84, 326-31 (2008).

In the current study, it is shown that the CYP2C9*5, *6, *8, and *11 alleles contribute to the variability in warfarin dose requirements beyond that of the CYP2C9*2 and *3 alleles and VKORC1 −1639G>A genotype among African Americans. Together, the CYP2C9*2, *3, *5, *6, *8, and *11 alleles; VKORC1 −1639G>A genotype; and clinical factors explained 36% of the variability in dose requirements in this population. In comparison, a model without the CYP2C9*5, *6, *8, and *11 alleles explained 30% of the variability. These data suggest that including the CYP2C9*5, *6, *8, and *11 alleles will improve the predictive ability of warfarin dosing algorithms for African Americans.

Consistent with our data for CYP2C9, Limdi et al., Pharmacogenomics 9, 511-26 (2008), previously reported lower warfarin doses among African Americans with a variant CYP2C9*2, *3, *5, *6, or *11 allele. The CYP2C9*8 allele was not included in their analysis. While the CYP2C9*2 and *3 alleles are the predominant CYP2C9 alleles in Caucasians, the CYP2C9*8 allele is more common in African Americans, with a reported frequency of 0.04 to 0.09 in African American and black African populations. Allabi, A. C., Gala, J. L. & Horsmans, Y. CYP2C9, CYP2C19, ABCB1 (MDR1) genetic polymorphisms and phenytoin metabolism in a Black Beninese population. Pharmacogenet Genomics 15, 779-86 (2005); Blaisdell, J. et al. Discovery of new potentially defective alleles of human CYP2C9. Pharmacogenetics 14, 527-37 (2004). The CYP2C9*5, *6, and *11 alleles also occur almost exclusively in African Americans. Importantly, over 3-times as many patients in this study carried a CYP2C9*5, *6, *8, or *11 allele (18%) versus a CYP2C9*2 or *3 allele (5%). Similar to data with the CYP2C9*2 and *3 alleles, patients with a CYP2C9*5, *6, *8, or *11 allele required significantly lower warfarin doses compared to CYP2C9*1 allele homozygotes. Higashi, M. K. et al. Association between CYP2C9 genetic variants and anticoagulation-related outcomes during warfarin therapy. Jama 287, 1690-8 (2002); Aithal, G. P., Day, C. P., Kesteven, P. J. & Daly, A. K. Association of polymorphisms in the cytochrome P450 CYP2C9 with warfarin dose requirement and risk of bleeding complications. Lancet 353, 717-9 (1999). The data suggest that neglecting to genotype and account for the CYP2C9*5, *6, *8, and *11 alleles when dosing warfarin could result in overdosing a significant portion of African Americans.

While previous investigators have reported reductions in enzyme activity with the CYP2C9*5, *6, and *11 alleles, data on the functional effects of the CYP2C9*8 allele are conflicting. In particular, investigation of catalytic activity toward tolbutamide in a recombinant system demonstrated increased activity with the CYP2C9*8 allele. Blaisdell, J. et al. Discovery of new potentially defective alleles of human CYP2C9. Pharmacogenetics 14, 527-37 (2004). In contrast, a clinical study using phenytoin as a phenotyping probe showed reduced urinary excretion of phenytoin metabolite with the CYP2C9*8 allele. Id; Allabi, A. C., Gala, J. L. & Horsmans, Y. CYP2C9, CYP2C19, ABCB1 (MDR1) genetic polymorphisms and phenytoin metabolism in a Black Beninese population. Pharmacogenet Genomics 15, 779-86 (2005). These disparate findings could be secondary to substrate-dependent activity of the CYP2C9*8 allele. Id. To Applicants' knowledge, there are no studies of the CYP2C9*8 allele using warfarin as a phenotyping probe. Alternatively, the CYP2C9*8 allele may be in linkage disequilibrium with another variant that causes reduced catalytic activity. In this regard, the R150H SNP has been linked to two promoter region SNPs, −1766T>C and −1188T>C, in a previous study. Blaisdell, J. et al. Discovery of new potentially defective alleles of human CYP2C9. Pharmacogenetics 14, 527-37 (2004). We observed lower dose requirements with the CYP2C9*8 allele suggesting that the *8 allele, or an allele in linkage disequilibrium with the CYP2C9*8 allele, is associated with decreased CYP2C9 metabolism of warfarin.

The variability in warfarin dose requirements explained by the VKORC1 −1639G>A or 1173A>G genotype is approximately 18 to 25% among Caucasians, 30% among Asians, but only about 5% in African Americans (7% in the current study). Schelleman, H. et al. Warfarin response and vitamin K epoxide reductase complex I in African Americans and Caucasians. Clin Pharmacol Ther 81, 742-7 (2007); Limdi, N. A. et al. VKORC1 polymorphisms, haplotypes and haplotype groups on warfarin dose among African-Americans and European-Americans. Pharmacogenomics 9, 1445-58 (2008); Veenstra, D. L. et al. Association of Vitamin K epoxide reductase complex 1 (VKORC1) variants with warfarin dose in a Hong Kong Chinese patient population. Pharmacogenet Genomics 15, 687-91 (2005); Gage, B. F. et al. Use of pharmacogenetic and clinical factors to predict the therapeutic dose of warfarin. Clin Pharmacol Ther 84, 326-31 (2008). Consistent with previous reports, the VKORC1 −4451 C>A, 497T>G, and 3730G>A SNPs provided no additional contribution to warfarin dose requirements in our study. Gage, B. F. et al. Use of pharmacogenetic and clinical factors to predict the therapeutic dose of warfarin. Clin Pharmacol Ther 84, 326-31 (2008); Limdi, N. A. et al. VKORC1 polymorphisms, haplotypes and haplotype groups on warfarin dose among African-Americans and European-Americans. Pharmacogenomics 9, 1445-58 (2008). Previous studies also show that VKORC1 haplotype is no more informative of warfarin dose requirements than either the −1639G>A or 1173C>T SNP. Id; Rieder, M. J. et al. Effect of VKORC1 haplotypes on transcriptional regulation and warfarin dose. N Engl J Med 352, 2285-93 (2005).

In contrast to previous data in Caucasians, there was no association between the CYP4F2 V433M variant and warfarin dose in our African American cohort. However, this is not inconsistent with the low minor allele frequency in the study population. In particular, the minor 433M allele frequency is approximately 25 to 30% in Caucasian and Asian populations, but only 6 to 7% in African Americans. Caldwell, M. D. et al. CYP4F2 genetic variant alters required warfarin dose. Blood 111, 4106-12 (2008). The 433M/M genotype is associated with lower CYP4F2 protein concentration and has been correlated with higher warfarin dose requirements compared to the V/V genotype in 3 independent Caucasian cohorts. Id. This association was confirmed in a recent genome wide association study in Swedish patients (see Takeuchi, F. et al. A genome-wide association study confirms VKORC1, CYP2C9, and CYP4F2 as principal genetic determinants of warfarin dose. PLoS Genet 5, e1000433 (2009).) and in cohorts of Spanish (see Perez-Andreu, V. et al. Pharmacogenetic relevance of CYP4F2 V433M polymorphism on acenocoumarol therapy. Blood 113, 4977-9 (2009) and Italian (see Borgiani, P. et al. CYP4F2 genetic variant (rs2108622) significantly contributes to warfarin dosing variability in the Italian population. Pharmacogenomics 10, 261-6 (2009) patients. A recent study showed that CYP4F2 catalyzes formation of a hydroxyvitamin K₁ metabolite from vitamin K₁, thus decreasing the concentration of vitamin K₁ available for reduction to vitamin KH₂, a necessary co-factor for hydroxylation and activation of vitamin K-dependent clotting factors. McDonald, M. G., Rieder, M. J., Nakano, M., Hsia, C. H. & Rettie, A. E. CYP4F2 Is a Vitamin K1 Oxidase: an Explanation for Altered Warfarin Dose in Carriers of the V433M Variant. Mol Pharmacol 75, 1337-46 (2009). Given the role of CYP4F2 on vitamin K₁ metabolism, it is possible that ethnic differences in dietary vitamin K₁ intake contributed to the disparate findings. However, we only assessed vitamin K intake in a subset of patients included in this study (data not reported), and thus can make no conclusions regarding the interaction between diet, genotype, and warfarin dose.

While several groups have reported associations between the APOE genotype and warfarin dose requirements, the data are conflicting. For example, the c4 allele was associated with higher therapeutic doses among African American (see Absher, R. K., Moore, M. E. & Parker, M. H. Patient-specific factors predictive of warfarin dosage requirements. Ann Pharmacother 36, 1512-7 (2002)) and Swedish (see Wadelius, M. et al. Association of warfarin dose with genes involved in its action and metabolism. Hum Genet 121, 23-34 (2007)) patients, but lower doses requirements in a cohort from the United Kingdom. Sconce, E. A., Daly, A. K., Khan, T. I., Wynne, H. A. & Kamali, F. APOE genotype makes a small contribution to warfarin dose requirements. Pharmacogenet Genomics 16, 609-11 (2006). No association between the APOE genotype and warfarin dose was observed in a Caucasian cohort from the U.S. (see Kimmel, S. E. et al. Apolipoprotein E genotype and warfarin dosing among Caucasians and African Americans. Pharmacogenomics J (2007)) or in either an Italian (see Kohnke, H., Scordo, M. G., Pengo, V., Padrini, R. & Wadelius, M. Apolipoprotein E (APOE) and warfarin dosing in an Italian population. Eur J Clin Pharmacol 61, 781-3 (2005)) or Asian (see Lal, S. et al. Influence of APOE genotypes and VKORC1 haplotypes on warfarin dose requirements in Asian patients. Br J Clin Pharmacol 65, 260-4 (2008)) patient population. In contrast to a previous study in African Americans (see Kimmel, S. E. et al. Apolipoprotein E genotype and warfarin dosing among Caucasians and African Americans. Pharmacogenomics J (2007)), there was no association between APOE genotype and warfarin dose in our population. Given the inconsistent data to date, further studies are necessary to delineate the role of the APOE genotype on warfarin dose.

Age, body size, and cerebrovascular disease were the only clinical factors associated with warfarin dose requirements in our population on regression analysis. Similarly, most other studies assessing factors associated with warfarin dose have noted age and body size (but not gender) to be important determinants of warfarin dose requirements. Aquilante, C. L. et al. Influence of coagulation factor, vitamin K epoxide reductase complex subunit 1, and cytochrome P450 2C9 gene polymorphisms on warfarin dose requirements. Clin Pharmacol Ther 79, 291-302 (2006); Klein, T. E. et al. Estimation of the warfarin dose with clinical and pharmacogenetic data. N Engl J Med 360, 753-64 (2009); Gage, B. F. et al. Use of pharmacogenetic and clinical factors to predict the therapeutic dose of warfarin. Clin Pharmacol Ther 84, 326-31 (2008); Sconce, E. A. et al. The impact of CYP2C9 and VKORC1 genetic polymorphism and patient characteristics upon warfarin dose requirements: proposal for a new dosing regimen. Blood 106, 2329-33 (2005); Wadelius, M. et al. Association of warfarin dose with genes involved in its action and metabolism. Hum Genet 121, 23-34 (2007); Huang, S. W. et al. Validation of VKORC1 and CYP2C9 genotypes on interindividual warfarin maintenance dose: a prospective study in Chinese patients. Pharmacogenet Genomics 19, 226-34 (2009), The mechanism underlying the association between cerebrovascular disease and warfarin dose is unclear. One possibility is that medications commonly prescribed to those with cerebrovascular disease may interact with warfarin. However, this and any other potential mechanisms require exploration.

A large portion of the variability in warfarin dose among African Americans was unexplained by our model and is likely due to other genetic, clinical, or dietary factors. Previous studies in predominately Caucasian populations have examined the correlation between warfarin dose-response and the calumenin, γ-glutamyl carboxylase, and microsomal epoxide hydrolase genes, among others, with varying results. Caldwell, M. D. et al. Evaluation of genetic factors for warfarin dose prediction. Clin Med Res 5, 8-16 (2007); Wadelius, M. et al. Association of warfarin dose with genes involved in its action and metabolism. Hum Genet 121, 23-34 (2007); Herman, D., Peternel, P., Stegnar, M., Breskvar, K. & Dolzan, V. The influence of sequence variations in factor VII, gamma-glutamyl carboxylase and vitamin K epoxide reductase complex genes on warfarin dose requirement. Thromb Haemost 95, 782-7 (2006); Loebstein, R. et al. Common genetic variants of microsomal epoxide hydrolase affect warfarin dose requirements beyond the effect of cytochrome P450 2C9. Clin Pharmacol Ther 77, 365-72 (2005); Vecsler, M. et al. Combined genetic profiles of components and regulators of the vitamin K-dependent gamma-carboxylation system affect individual sensitivity to warfarin. Thromb Haemost 95, 205-11 (2006); Rieder, M. J., Reiner, A. P. & Rettie, A. E. Gamma-glutamyl carboxylase (GGCX) tagSNPs have limited utility for predicting warfarin maintenance dose. J Thromb Haemost 5, 2227-34 (2007). Whether these genes influence warfarin dose requirements in African Americans has not been reported and requires study. Genome wide association studies in African Americans may also reveal novel gene variations influencing warfarin dose requirements in this population. Recent data suggests that renal function may influence warfarin dose requirements, and this too should be examined as a predictor of warfarin dose. Limdi, N. A. et al. Kidney function influences warfarin responsiveness and hemorrhagic complications. J Am Soc Nephrol 20, 912-21 (2009).

In conclusion, Applicants' study found that the combination of clinical and genetic factors, including the CYP2C9 variants that occur predominately in African Americans and the VKORC1 −1639G>A genotype contribute to the variability in warfarin dose requirements among African Americans. Together, these factors explained 36% of the inter-patient variability in warfarin dose requirements. In contrast, there was no association between CYP4F2 or APOE genotype and warfarin dose requirements. Our data suggest that the inclusion of the CYP2C9*5, *6, *8, and *11 alleles in warfarin dosing algorithms, in addition to the CYP2C9*2 and *3 alleles and VKORC1 −1639G>A genotype, may improve the predictive ability of the algorithm for African Americans. Formal algorithm development to include these variants may be accomplished as was done by the International Warfarin Pharmacogenomics Consortium. Klein, T. E. et al. Estimation of the warfarin dose with clinical and pharmacogenetic data. N Engl J Med 360, 753-64 (2009).

### Example 1

### Study Population

Patients were enrolled from the pharmacist-managed anticoagulation clinics at the University of Illinois Medical Center at Chicago and at the University of Florida in Gainesville. The inclusion criteria were age ≧18 years, African American race by self report, and treatment with a stable dose of warfarin, defined as the same dose for at least 3 consecutive clinic visits, as previously described. Aquilante, C. L. et al. Influence of coagulation factor, vitamin K epoxide reductase complex subunit 1, and cytochrome P450 2C9 gene polymorphisms on warfarin dose requirements. Clin Pharmacol Ther 79, 291-302 (2006); Momary, K. M. et al. Factors influencing warfarin dose requirements in African-Americans. Pharmacogenomics 8, 1535-44 (2007). Patients with a history of liver dysfunction or serum transaminase levels greater than 3 times the upper limit of normal were excluded.

### Example 2

### Data Collection

After obtaining written informed consent and authorization for medical record review, a buccal cell sample was collected for genetic analysis, as previously described. Andrisin, T. E., Humma, L. M. & Johnson, J. A. Collection of genomic DNA by the noninvasive mouthwash method for use in pharmacogenetic studies. Pharmacotherapy 22, 954-60 (2002). Demographic, clinical, and social history were assessed through patient interview and review of the medical record. This study was approved by the Institutional Review Board at each institution.

### Example 3

### Genotyping

Genomic DNA was isolated from buccal cells using a commercially available kit (PureGene,® Qiagen, Valencia, Calif.) according to kit manufacturer instructions. The CYP2C9 R144C (*2), 1359L (*3) and D360E (*5) alleles and VKORC1 −1639G>A genotype were determined as previously described. Aquilante, C. L. et al. Influence of coagulation factor, vitamin K epoxide reductase complex subunit 1, and cytochrome P450 2C9 gene polymorphisms on warfarin dose requirements. Clin Pharmacol Ther 79, 291-302 (2006); Hruska, M. W., Frye, R. F. & Langaee, T. Y. Pyrosequencing method for genotyping cytochrome P450 CYP2C8 and CYP2C9 enzymes. Clin Chem 50, 2392-5 (2004). The VKORC1 −4451A>C, 497G>T, and 3730A>G genotypes, and APOE 112C>T and 158C>T genotypes were determined by PCR and pyrosequencing. PCR and sequencing primers used were per Table 6.

TABLE 6
PCR and pyrosequencing primers

| SNP | Primers (5′ to 3′) |
|---|---|
| VKORC1 −4451A>C | PCR Forward: |
| | TCTTGGAGTGAGGAAGGCAAT |
| | (SEQ ID. NO. 1) |
| | PCR Reverse: |
| | biotin-GACAGGTCTGGACAACGTGG |
| | (SEQ ID. NO. 2) |
| | Sequencing: |
| | CTCAGGTGATCCA |
| | (SEQ ID. NO. 3) |
| VKORC1 497G>T | PCR Forward: |
| | biotin-GGATGCCAGATGATTATTCTGGAGT |
| | (SEQ ID. NO. 4) |
| | PCR Reverse: |
| | TCATTATGCTAACGCCTGGCC |
| | (SEQ ID. NO. 5) |
| | Sequencing: |
| | CAACACCCCCCTTC |
| | (SEQ ID. NO. 6) |
| VKORC1 3730A>G | PCR Forward: |
| | TACCCCCTCCTCCTGCCATA |
| | (SEQ ID. NO. 7) |
| | PCR Reverse: |
| | Biotin-CCAGCAGGCCCTCCACTC |
| | (SEQ ID. NO. 8) |
| | Sequencing: |
| | TCCTCCTGCCATACC |
| | (SEQ ID. NO. 9) |
| APOE 112C>T | PCR Forward: |
| | Biotin-GCGGACATGGAGGACGTG |
| | (SEQ ID. NO. 10) |
| | PCR Reverse: |
| | TACACTGCCAGGCGCTTCT |
| | (SEQ ID. NO. 11) |
| | Sequencing: |
| | ACTGCACCAGGCGGC |
| | (SEQ ID. NO. 12) |
| APOE 158C>T | PCR Forward: |
| | CTCCGCGATGCCGATGAC |
| | (SEQ ID. NO. 13) |
| | PCR Reverse: |
| | Biotin-CCCCGGCCTGGTACACTG |
| | (SEQ ID. NO. 14) |
| | Sequencing: |
| | CGATGACCTGCAGAAG |
| | (SEQ ID. NO. 15) |

Each PCR reaction consisted of 25 μl of HotStarTaq™ Master Mix (Qiagen), primers (25 μmol), 15 μl of H₂O, and 20-100 ng of DNA. Thermocycling consisted of denaturation for 15 minutes at 95° C., followed by 40 cycles of denaturation at 94° C. for 30 seconds, annealing at 61° C. (67° C. for APOE c.112C>T) for 30 seconds, and extension at 72° C. for 1 minute, with a final extension of 72° C. for 10 minutes.

Prior to determination of additional genotypes, genomic DNA was amplified by the whole genome amplification (WGA) technique, using the REPLI-g midi kit (Qiagen). Dean, F. B. et al. Comprehensive human genome amplification using multiple displacement amplification. Proc Natl Acad Sci USA 99, 5261-6 (2002) The fidelity of amplification was verified by comparing genotypes for CYP2C9*2, *3, *5 and VKORC1 −1639G>A determined before and after WGA. Genotyping for the CYP4F2 V433M polymorphism and CYP2C9*6 and *11 alleles was conducted at Osmetech Molecular Diagnostics (Pasadena, Calif.) using the eSensor® Warfarin Sensitivity Test. Reed, M. R. & Coty, W. A. in Microarrays: Preparation, Detection Methods, Data Analysis, and Applications (eds. Dill, K., Liu, R. & Grodzinski, P.) 247-60 (Springer-Verlag/Kluwer, 2009). The CYP2C9*8 genotype was determined by bi-directional DNA sequencing at Agencourt Biosciences after uniplex PCR amplification performed by Osmetech Molecular Diagnostics. All genotypes were assigned by investigators blinded to warfarin dose.

### Example 4

### Data Analysis

Data are expressed as numbers (percentages), mean±SD, or median (inter-quartile range). Height and weight were used to calculate body surface area (BSA). Average INR was calculated from the mean of INR values from the enrollment visit and the 2 previous visits for which the warfarin dose was stable. Hardy Weinberg Equilibrium (HWE) assumption was tested by χ² analysis. Median weekly warfarin dose requirements were compared between genotype groups by the Mann Whitney U test. The variant CYP2C9*2, *3, *5, *6, and *11 alleles were initially combined into one group for univariate dose comparisons, given their low frequency in African Americans and previous evidence that CYP2C9 enzyme activity is reduced in the presence of each allele. Dickmann, L. J. et al. Identification and functional characterization of a new CYP2C9 variant (CYP2C9*5) expressed among African Americans. Mol Pharmacol 60, 382-7 (2001); Allabi, A. C., Gala, J. L. & Horsmans, Y. CYP2C9, CYP2C19, ABCB1 (MDR1) genetic polymorphisms and phenytoin metabolism in a Black Beninese population. Pharmacogenet Genomics 15, 779-86 (2005); Blaisdell, J. et al. Discovery of new potentially defective alleles of human CYP2C9. Pharmacogenetics 14, 527-37 (2004) Carriers of a variant CYP2C9*8 allele were initially analyzed separately because of uncertainty regarding the functional effects of this allele.

Warfarin dose was log transformed prior to further analysis to improve the normality of its distribution. The Pearson's correlation coefficient and Student's unpaired t-test were used to identify clinical factors associated with log-transformed warfarin dose requirements. Variables tested were age, gender, BSA, target INR, history of venous thromboembolism, heart failure, prior ischemic stroke or transient ischemic attack, active cancer, current smoker, and use of amiodarone, aspirin, simvastatin, or either phenytoin or carbamazepine.

Stepwise regression was used to determine the clinical and genetic variables jointly associated with warfarin dose requirements. Clinical variables that were potentially associated with therapeutic warfarin dose by univariate analysis (p<0.10) and all genotypes were tested in the model. Genotypes were entered into the model first. A dominant model was used for the VKORC1 3730A>G (GG versus non-GG) and APOE (ε4 carriers versus non-ε4 carrier) genotypes. An additive model was used for all other genotypes, with the number of variant alleles coded as “0”, “1”, or “2.” Variables that were significant predictors of warfarin dose (p<0.05) were retained in the model. The adjusted R² after entry of each variable provides an indication of the variable's contribution to the model. The final adjusted R² of the model indicates the joint contribution of all variables to the inter-patient variability in warfarin dose requirements.

All articles and documents referenced herein, as well as the references cited therein, are incorporated by reference for an understanding of the invention and are indicative of what the person of ordinary skill in the art knows or needs to know in order to appreciate and practice the invention using no more than routine experimentation.

The methods and compositions described illustrate preferred embodiments, are exemplary, and are not intended as limitations on the scope of the invention. Certain modifications and other uses will be apparent to those skilled in the art, and are encompassed within the spirit of the invention as defined by the scope of the claims.

The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description, including tables and claims.

1. A method of administering warfarin or a warfarin derivative to a subject in need thereof, comprising:
determining whether a subject possesses a CYP2C9*8 allele and, if that subject possesses said allele,
administering an amount of warfarin or warfarin derivative more appropriate than would be administered to a homozygous wild type patient not bearing said allele.

2. The method of claim 1 wherein said amount is a lower amount.

3. The method of claim 1 wherein said subject is African-American, black African, or of black African descent.

4. The method of claim 1 wherein said homozygous wild type is *1/*1.

5. The method of claim 1 wherein said subject is homozygous for said allele.

6. The method of claim 1 wherein said subject is heterozygous for said allele.

7. The method of claim 2 wherein the genotype of said subject is *5/*8.

8. The method of claim 2 wherein the genotype of said subject is *8/*11.

9. The method of claim 1 wherein the genotype of said subject is *8 combined with one or more other alleles associated with reduced metabolism of warfarin.

10. The method of claim 9 wherein said some one or more other alleles associated with reduced metabolism of warfarin is selected from the group consisting of *2, *3, *5, *6 and *11.

11. The method of claim 9 wherein the *8 genotype is combined with each of the genotypes for *2, *3, *5, *6, and *11.

12. The method of claim 9 wherein the *8 genotype is combined with each of the genotypes for *5, *6, and *11.

13. The method of claim 1 wherein said administering step comprises administering between about 55% and 85% of the warfarin dose that would be administered to a homozygous wild type patient.

14. The method of claim 1 wherein said administering step comprises administering to said subject between about 25 mg/week and 40 mg/week.

15. A warfarin dosing algorithm, said warfarin dosing algorithm comprising one more mathematical operations that consider CYP2C9*8 genotype in calculating, predicting, and/or prescribing warfarin dosage to a patient, and wherein said calculating, predicting, and/or prescribing comprises a lower amount of warfarin relative to a homozygous wild type CYP2C9 genotype.

16. A method of identifying a subject with increased sensitivity to warfarin comprising:
identifying a CYP2C9*8 polymorphism in said subject, wherein the presence of said polymorphism is indicative of a patient with increased sensitivity to warfarin relative to a subject having the corresponding wild-type allele.

17. The method of claim 16, wherein said identifying comprises detecting the polymorphism in the DNA of said subject.

18. The method of claim 16, wherein said identifying comprises detecting the polymorphism in the CYP2C9 gene product.


SEQUENCE LISTING (15 sequences)

SEQ ID NO: 1 - DNA, 21 bp, Artificial Sequence
  Primer
tcttggagtgaggaaggcaat

SEQ ID NO: 2 - DNA, 20 bp, Artificial Sequence
  Primer
gacaggtctggacaacgtgg

SEQ ID NO: 3 - DNA, 13 bp, Artificial Sequence
  Primer
ctcaggtgatcca

SEQ ID NO: 4 - DNA, 25 bp, Artificial Sequence
  Primer
ggatgccagatgattattctggagt

SEQ ID NO: 5 - DNA, 21 bp, Artificial Sequence
  Primer
tcattatgctaacgcctggcc

SEQ ID NO: 6 - DNA, 14 bp, Artificial Sequence
  Primer
caacaccccccttc

SEQ ID NO: 7 - DNA, 20 bp, Artificial Sequence
  Primer
taccccctcctcctgccata

SEQ ID NO: 8 - DNA, 18 bp, Artificial Sequence
  Primer
ccagcaggccctccactc

SEQ ID NO: 9 - DNA, 15 bp, Artificial Sequence
  Primer
tcctcctgccatacc

SEQ ID NO: 10 - DNA, 18 bp, Artificial Sequence
  Primer
gcggacatggaggacgtg

SEQ ID NO: 11 - DNA, 19 bp, Artificial Sequence
  Primer
tacactgccaggcgcttct

SEQ ID NO: 12 - DNA, 15 bp, Artificial Sequence
  Primer
actgcaccaggcggc

SEQ ID NO: 13 - DNA, 18 bp, Artificial Sequence
  Primer
ctccgcgatgccgatgac

SEQ ID NO: 14 - DNA, 18 bp, Artificial Sequence
  Primer
ccccggcctggtacactg

SEQ ID NO: 15 - DNA, 16 bp, Artificial Sequence
  Primer
cgatgacctgcagaag